(19) United States
(12) Patent Application Publication
(10) Pub. No.: US 2020/0155012 A1
(43) Pub. Date: May 21, 2020

(54) BLOOD OXYGEN SENSING DEVICE

(71) Applicant: ACARE TECHNOLOGY CO., LTD., New Taipei City (TW)

(72) Inventor: Lin-Ta CHENG, Kaohsiung City (TW)

(21) Appl. No.: 16/450,103

(22) Filed: Jun. 24, 2019

(30) Foreign Application Priority Data
Nov. 19, 2018 (TW) 107215676

(51) Int. Cl. A61B 5/0205; A61B 5/1455; A61B 5/00

(52) U.S. Cl. CPC A61B 5/0205; A61B 5/14551; A61B 5/02116; A61B 5/742; A61B 5/7278

(57) ABSTRACT

A blood oxygen sensing device includes a sensor, a processing module and a display module. The sensor emits first and second sensing light sources to a to-be-sensed target alternately, and generate first and second sensing signals. The processing module generates first and second sensing signal base lines, generates a heartbeat pulse signal according to the first and second sensing signals matching a first preset range, records a pulse amplitude signal and a DC signal of the heartbeat pulse signal matching a second preset range, and generates a blood oxygen value and a pulse value according to the pulse amplitude signal and the DC signal. The processing module chronologically sorts the blood oxygen values and the pulse values in a sequential order, respectively. The display module is connected to the processing module and receives and displays the blood oxygen value and the pulse value updated.

### CROSS-REFERENCE TO RELATED APPLICATION

This application claims the benefit of Taiwan Patent Application No. 107215676, filed on Nov. 19, 2018, in the Taiwan Intellectual Property Office, the disclosure of which is incorporated herein in its entirety by reference.

### BACKGROUND OF THE INVENTION

### 1. Field of the Invention

The present invention relates to a sensing device, more particularly to a blood oxygen sensing device.

### 2. Description of the Related Art

Generally, a conventional blood oxygen sensing machine must face the following problems.

First, vessel density is different when a measurement position of a probe is different.

Secondly, tightness between a fixture and a blood oxygen sensing machine is possibly changed subject to user motion.

Thirdly, ambient light is changed when the user moves to different environment.

Fourthly, sweat or dirt possibly affects signal strength of the blood oxygen sensing machine.

Fifthly, a light source and a sensing component of the blood oxygen sensing machine are aged after a long-term use.

Sixthly, a reminding signal must be outputted when the blood oxygen sensing machine does not sense a correct signal.

The above-mentioned problems possibly impact accuracy of the blood oxygen sensing machine, and how to solve these problems is a key issue in the industry.

### SUMMARY OF THE INVENTION

An objective of the present invention is to provide a blood oxygen sensing device to solve the conventional problems.

In order to achieve the objective, the present invention provides a blood oxygen sensing device comprising a sensor, a processing module and a display module. The sensor is configured to continuously emit a first sensing light source and a second sensing light source to a to-be-sensed target alternately, and continuously generate a first sensing signal and a second sensing signal alternately. The processing module is connected to the sensor, and is configured to continuously receive the first sensing signal and the second sensing signal. A first sensing signal base line and a second sensing signal base line are continuously generated according to the first sensing signal and the second sensing signal. The processing module also continuously determines whether or not both of the first sensing signal base line and the second sensing signal base line match the first preset range. A heartbeat pulse signal is also continuously generated according to the first sensing signal and the second sensing signal and the heartbeat pulse signal matches the first preset range. A blood pulse amplitude of the heartbeat pulse signal is also continuously determined to match a second preset range, and a pulse amplitude signal and a DC signal of the heartbeat pulse signal matching the second preset range are continuously recorded. A blood oxygen value and a pulse value are continuously generated according to the pulse amplitude signal and the DC signal, and the blood oxygen values and the pulse values are chronologically sorted in a sequential order, respectively. The display module is connected to the processing module, and is configured to receive and display the updated blood oxygen value and the pulse value.

In a preferred embodiment of the present invention, the blood oxygen sensing device further includes a driving module connected to the sensor and the processing module. The driving module is configured to drive the sensor to emit the first sensing light source and the second sensing light source, and receive the first sensing signal and the second sensing signal, and transmit the first sensing signal and the second sensing signal to the processing module.

In a preferred embodiment of the present invention, the processing module generates an adjustment signal in response to the first sensing signal and the second sensing signal which do not match the first preset range. The adjustment signal is transmitted to the driving module, and the driving module adjusts a driving parameter according to the adjustment signal, which generates a driving signal according to the driving parameter, and transmits the driving signal to the sensor. The sensor emits the first sensing light source and the second sensing light source according to the driving signal.

In a preferred embodiment of the present invention, the processing module records one of the pulse amplitude signals as an initial value, and generates the adjustment signal in response to the pulse amplitude signal after the pulse amplitude signal is consecutively drifted from the pulse amplitude signal as the initial value in a consecutive predetermined period.

In a preferred embodiment of the present invention, the processing module records one of the DC signals as an initial value, and generates the adjustment signal in response to the DC signal after the DC signal is consecutively drifted from the DC signal as the initial value in a predetermined period.

In a preferred embodiment of the present invention, the blood oxygen sensing device further comprises an alert unit connected to the processing module. The processing module generates the abnormal signal in response to both of the first sensing signal and the second sensing signal not matching the first preset range, transmits the abnormal signal to the alert unit, and the alert unit generates an alert signal in response to the abnormal signal.

The blood oxygen sensing device according to claim 1, further comprising an alert unit connected to the processing module, wherein the processing module generates an abnormal signal in response to the heartbeat pulse signal not matching the second preset range, and transmits the abnormal signal to the alert unit, and the alert unit generates an alert signal in response to the abnormal signal.

In a preferred embodiment of the present invention, when the pulse value matches a third preset range, the processing module calculates a blood oxygen correlation coefficient according to the pulse amplitude signal and the DC signal, and inputs the blood oxygen correlation coefficient into a blood oxygen experience equation, to calculate a blood oxygen value.

In a preferred embodiment of the present invention, after a blood oxygen median among the plurality of continuously-generated blood oxygen values is determined, a pulse median among the plurality of continuously-generated pulse values is determined. When one of the blood oxygen values minus the blood oxygen median is higher than a blood oxygen setting threshold, the blood oxygen value is replaced with the blood oxygen median, or, when one of the pulse values minus the pulse median is higher than a pulse setting threshold, the pulse value is replaced with the pulse median.

According to above contents, the blood oxygen sensing device of the present invention can automatically adjust the first sensing light source and the second sensing light source, and can notice the user to adjust the wearing position of the sensor when the first sensing signal and the second sensing signal are of poor quality.

### BRIEF DESCRIPTION OF THE DRAWINGS

The structure, operating principle and effects of the present invention will be described in detail by way of various embodiments which are illustrated in the accompanying drawings.

FIG. 1 is a first block diagram of a blood oxygen sensing device of the present invention.

FIG. 2 is a second block diagram of a blood oxygen sensing device of the present invention.

FIG. 3 is a third block diagram of a blood oxygen sensing device of the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

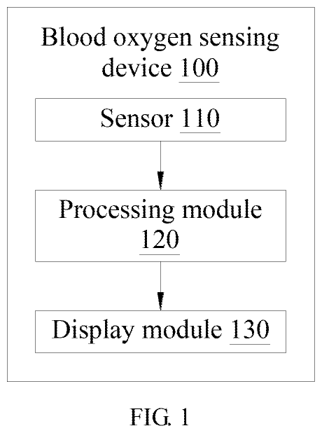

Please refer to FIG. 1, which is a first block diagram of a blood oxygen sensing device of the present invention. As shown in FIG. 1, a blood oxygen sensing device 100 includes a sensor 110, a processing module 120 and a display module 130.

The sensor 110 can continuously emit a first sensing light source and a second sensing light source on a to-be-sensed target alternately, and to continuously generate a first sensing signal and a second sensing signal alternately.

The processing module 120 is connected to the sensor 110, and continuously receives the first sensing signal and the second sensing signal, continuously generates a first sensing signal base line and a second sensing signal base line according to the first sensing signal and the second sensing signal, continuously determines whether the first sensing signal base line and the second sensing signal base line match a first preset range, continuously generates a heartbeat pulse signal according to the first sensing signal and the second sensing signal which both match the first preset range, continuously determines whether a blood pulse amplitude of the heartbeat pulse signal matches a second preset range, continuously records a pulse amplitude signal and a DC signal of the heartbeat pulse signal matching the second preset range, continuously generates a blood oxygen value and a pulse value according to the recorded pulse amplitude signal and the DC signal, and chronologically sorts the blood oxygen values and the pulse values in a sequential order, respectively.

The display module 130 is connected to the processing module 120, and receives and displays the updated blood oxygen value and the pulse value.

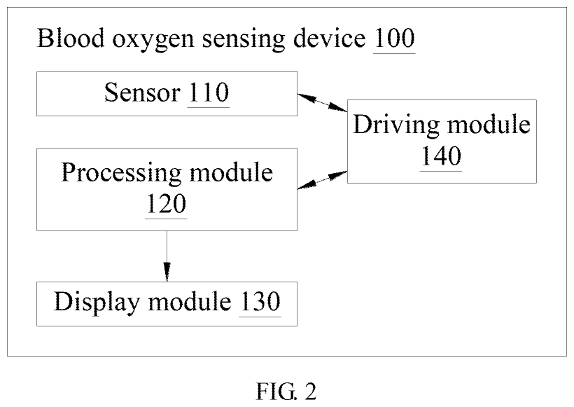

Please refer to FIG. 2, which is a second block diagram of a blood oxygen sensing device of the present invention. As shown in FIG. 2, the blood oxygen sensing device 100 can comprise a driving module 140 which is connected to the sensor 110 and the processing module 120. The driving module 140 can drive the sensor 110 to emit the first sensing light source and the second sensing light source, and receive the first sensing signal and the second sensing signal, and transmit the first sensing signal and the second sensing signal to the processing module 120.

The processing module 120 can generate an adjustment signal in response to the first sensing signal and the second sensing signal which both do not match the first preset range, and transmit the adjustment signal to the driving module 140. The driving module 140 can adjust a driving parameter according to the adjustment signal and generate a driving signal, according to the driving parameter, and transmit the driving signal to the sensor 110. The sensor 110 can emit the first sensing light source and the second sensing light source according to the driving signal.

The sensor 110 can comprise a first light-emitting unit configured to generate the first sensing light source, a second light-emitting unit configured to generate the second sensing light source, and an amplifier. In a process of automatically adjusting the sensor 110, the driving parameters, including current of the first light-emitting unit, current of the second light-emitting unit, and a magnification of the amplifier, are initialized first, and light intensities of the first light-emitting unit and the second light-emitting unit are captured to calculate average values, respectively. When the average values are higher than a setting threshold, the automatic adjustment process is completed. When the average values are not higher than the setting threshold, the currents of the first light-emitting unit and the second light-emitting unit are increased. When the currents of the first light-emitting unit and the second light-emitting unit are not higher than a current setting threshold, the currents of the first light-emitting unit and the second light-emitting unit are initialized and the magnification of the amplifier is increased. When the currents of the first light-emitting unit and the second light-emitting unit are higher than the current setting threshold, the average values of the first light-emitting unit and the second light-emitting unit are continuously calculated, respectively.

The processing module 120 can record one of the pulse amplitude signals as an initial value, and generate the adjustment signal in response to the pulse amplitude signal after the pulse amplitude signal is consecutively drifted from the pulse amplitude signal as the initial value in a predetermined period.

On the other hand, the processing module 120 can record one of the DC signals as an initial value, and generate the adjustment signal in response to the DC signal after the DC signal is consecutively drifted from the DC signal as the initial value in a predetermined period. Preferably, the predetermined period is 4 seconds.

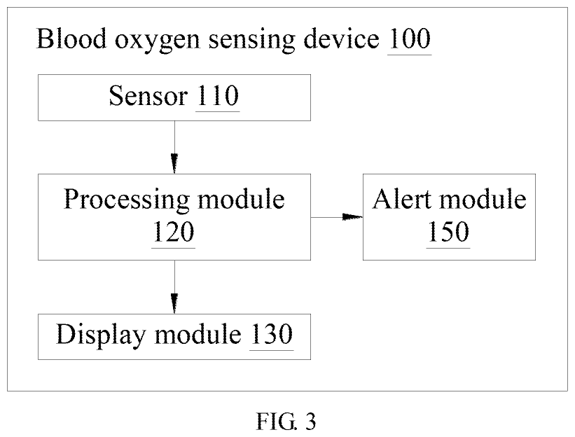

Please refer to FIG. 3, which is a third block diagram of a blood oxygen sensing device of the present invention. As shown in FIG. 3, the blood oxygen sensing device 100 can comprise an alert unit 150 connected to the processing module 120. The processing module 120 can generate an abnormal signal in response to both of the first sensing signal and the second sensing signal not matching the first preset range, and transmit the abnormal signal to the alert unit 150. The alert unit 150 can generate an alert signal in response to the abnormal signal.

On the other hand, the processing module 120 can generate the abnormal signal in response to the heartbeat pulse signal not matching the second preset range, and transmit the abnormal signal to the alert unit 150. The alert unit 150 can generate the alert signal in response to the abnormal signal.

When the pulse value matches the third preset range, the processing module 120 can calculate a blood oxygen correlation coefficient according to the pulse amplitude signal and the DC signal, and the blood oxygen correlation coefficient is inputted into a blood oxygen experience equation, such as the Beer-Lambert law, to calculate a blood oxygen value.

Furthermore, after a blood oxygen median among the plurality of continuously-generated blood oxygen values is determined, and a pulse median among the plurality of continuously-generated pulse values is determined, when one of the blood oxygen values minus the blood oxygen median is higher than a blood oxygen setting threshold, the blood oxygen value is replaced with the blood oxygen median, or, when one of the pulse values minus the pulse median is higher than a pulse setting threshold, the pulse value is replaced with the pulse median.

According to above contents, the blood oxygen sensing device of the present invention can automatically adjust the first sensing light source and the second sensing light source, and when the first sensing signal and the second sensing signal are of poor quality, the user can be noticed to adjust the wearing position of the sensor.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A blood oxygen sensing device, comprising:
a sensor configured to continuously emit a first sensing light source and a second sensing light source to a to-be-sensed target alternately, and continuously generate a first sensing signal and a second sensing signal alternately;
a processing module connected to the sensor, and configured to continuously receive the first sensing signal and the second sensing signal, and continuously generate a first sensing signal base line and a second sensing signal base line according to the first sensing signal and the second sensing signal, and continuously determine whether both of the first sensing signal base line and the second sensing signal base line match the first preset range, and continuously generate a heartbeat pulse signal according to the first sensing signal and the second sensing signal matching the first preset range, and continuously determine whether a blood pulse amplitude of the heartbeat pulse signal matches a second preset range, and continuously record a pulse amplitude signal and a DC signal of the heartbeat pulse signal matching the second preset range, and continuously generate a blood oxygen value and a pulse value according to the pulse amplitude signal and the DC signal, and chronologically sort the blood oxygen values and the pulse values in a sequential order, respectively; and
a display module connected to the processing module, and configured to receive and display the updated blood oxygen value and the pulse value.

2. The blood oxygen sensing device according to claim 1, further comprising a driving module connected to the sensor and the processing module, having the driving module configured to drive the sensor to emit the first sensing light source and the second sensing light source, receiving the first sensing signal and the second sensing signal, and transmitting the first sensing signal and the second sensing signal to the processing module.

3. The blood oxygen sensing device according to claim 2, wherein the processing module generates an adjustment signal in response to the first sensing signal and the second sensing signal which do not match the first preset range, and transmits the adjustment signal to the driving module, and the driving module adjusts a driving parameter according to the adjustment signal, and generates a driving signal according to the driving parameter, and transmits the driving signal to the sensor, and the sensor emits the first sensing light source and the second sensing light source according to the driving signal.

4. The blood oxygen sensing device according to claim 3, wherein the processing module records one of the pulse amplitude signals as an initial value, and generates the adjustment signal in response to the pulse amplitude signal after the pulse amplitude signal is consecutively drifted from the pulse amplitude signal as the initial value in a predetermined period.

5. The blood oxygen sensing device according to claim 3, wherein the processing module records one of the DC signals as an initial value, and generates the adjustment signal in response to the DC signal after the DC signal is consecutively drifted from the DC signal as the initial value in a predetermined period.

6. The blood oxygen sensing device according to claim 1, further comprising an alert unit connected to the processing module, wherein the processing module generates an abnormal signal in response to both of the first sensing signal and the second sensing signal not matching the first preset range, and transmits the abnormal signal to the alert unit, and the alert unit generates an alert signal in response to the abnormal signal.

7. The blood oxygen sensing device according to claim 1, further comprising an alert unit connected to the processing module, wherein the processing module generates an abnormal signal in response to the heartbeat pulse signal not matching the second preset range, and transmits the abnormal signal to the alert unit, and the alert unit generates an alert signal in response to the abnormal signal.

8. The blood oxygen sensing device according to claim 1, wherein when the pulse value matches a third preset range, the processing module calculates a blood oxygen correlation coefficient according to the pulse amplitude signal and the DC signal, and inputs the blood oxygen correlation coefficient into a blood oxygen experience equation, to calculate a blood oxygen value.

9. The blood oxygen sensing device according to claim 1, wherein after a blood oxygen median among the plurality of continuously-generated blood oxygen values is determined, and a pulse median among the plurality of continuously-generated pulse values is determined, when one of the blood oxygen values minus the blood oxygen median is higher than a blood oxygen setting threshold, the blood oxygen value is replaced with the blood oxygen median, or, when one of the pulse values minus the pulse median is higher than a pulse setting threshold, the pulse value is replaced with the pulse median.
